(19) United States
(12) Patent Application Publication
(10) Pub. No.: US 2025/0325257 A1
(43) Pub. Date: Oct. 23, 2025

(54) INGESTIBLE CAPSULE DEVICE

(71) Applicant: Eli Lilly and Company, Indianapolis, IN (US)

(72) Inventors: Pedro IRAZOQUI, Baltimore, MD (US); Sachin KADIAN, West Lafayette, IN (US); Sadid Rahman KHAN, Baltimore, MD (US); Trevor Donald MEYER, Baltimore, MD (US); Rahim RAHIMI, West Lafayette, IN (US); Vidhya SELVAMANI, Carrboro, NC (US); Mohit Singh VERMA, West Lafayette, IN (US)

(21) Appl. No.: 18/865,584

(22) PCT Filed: May 18, 2023
(86) PCT No.: PCT/US2023/022650

§ 371 Date: Nov. 13, 2024

Related U.S. Application Data
(60) Provisional application No. 63/344,090, filed on May 20, 2022.

(51) Int. Cl. A61B 10/00; A61B 5/1459

(52) U.S. Cl. CPC A61B 10/0038; A61B 5/1459; A61B 2010/0061; A61B 2562/162

(57) ABSTRACT

The techniques provide for non-invasive gastrointestinal sampling. In some embodiments, a device includes a capsule housing bounding a cavity, a sampling aperture formed in the capsule housing and providing fluid communication between the cavity and an exterior of the capsule housing, a luminescent substrate layer positioned within cavity, the luminescent substrate being configured to emit a luminescent light upon exposure to a sample fluid containing a luminescing trigger, and at least one additional substrate layer positioned within the cavity between the sampling aperture and the luminescent substrate, each of the at least one additional substrate layers being configured to chemically interact with the sample fluid. The device also includes a photodetector positioned within the cavity, the photodetector configured to detect the luminescent light, and a biodegradable coating closing the sampling aperture such that degradation of the biodegradable coating exposes the sampling aperture to permit fluid flow into the cavity.

### FIELD

Disclosed embodiments are related to ingestible capsule devices for detection and evaluation of gastrointestinal conditions and related methods of use.

### BACKGROUND

Development of tools that can accurately detect certain conditions within a gastrointestinal (GI) tract may facilitate medical advances, such as effective and accurate prediction and diagnosis of disease and disease progression. For example, inflammatory bowel diseases (IBD) are a group of intestinal disorders that cause chronic remitting and relapsing inflammation of the GI tract. Various factors may contribute to the growth of IBD, including the hyper-reactive response of the immune system, genetic variations in multiple genes, gut microbiota, and diet. Almost 6.8 million cases of IBD were reported globally in the year 2017. It is estimated that almost 1.6 million people in the United States alone suffer from two common types of IBD (Crohn's disease (CD) and ulcerative colitis (UC)). It can therefore be desirable to predict and/or diagnose IBD progression.

Conventional techniques for diagnosing IBD can include, for example, monitoring symptoms, endoscopies, colonoscopies, and capsule endoscopies. More recently, certain biomarkers have been correlated with the occurrence and relapse of IBD. Stool sampling techniques have been developed to detect these biomarkers in a patient's stool. However, there remains a need for more effectively and accurately diagnosing IBD and other GI conditions.

### SUMMARY

In some embodiments, a device for passive sampling of a gastrointestinal tract of a patient may comprise a capsule housing bounding a cavity, and a sampling aperture formed in the capsule housing. The sampling aperture may provide fluid communication between the cavity and an exterior of the capsule housing. The device may further comprise a luminescent substrate layer positioned within the cavity. The luminescent substrate may be configured to emit a luminescent light upon exposure to a sample fluid containing a luminescing trigger. The device may further include at least one additional substrate layer positioned within the cavity between the sampling aperture and the luminescent substrate. Each of the at least one additional substrate layers may be configured to chemically interact with the sample fluid. The device may also include a photodetector positioned within the cavity. The photodetector may be configured to detect the luminescent light. The device may further comprise a biodegradable coating closing the sampling aperture such that degradation of the biodegradable coating may expose the sampling aperture to permit fluid flow into the cavity.

In some embodiments, a method for detecting a biomarker in a gastrointestinal tract of a patient may be provided, the method comprising administering to a patient an ingestible device. The ingestible device may comprise a capsule housing bounding a cavity and a sampling aperture formed in the capsule housing and providing fluid communication between the cavity and an exterior of the capsule housing. A luminescent substrate may be positioned within the cavity. The luminescent substrate may be configured to emit a luminescent light upon exposure to a sample fluid containing a luminescing trigger. The luminescing trigger may be indicative of a presence of the biomarker. A photodetector may additionally be positioned within the cavity. The photodetector may be configured to detect the luminescent light and generate a detection signal. A wireless transmitter of the device may be configured to transmit a wireless signal based on the detection signal. The method may further comprise exposing the luminescent substrate to the sample fluid, receiving the wireless signal at a user device, and determining, based on the wireless signal, whether the biomarker is present in the sample fluid.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows a schematic internal view of an ingestible capsule device according to one embodiment;

FIG. 2 shows a schematic view of an ingestible capsule device operating within a GI tract of a patient according to one embodiment;

FIG. 3 shows a schematic view of various substrate layers and a photodetector of one embodiment;

FIG. 4A shows a plot of luminescent intensity during a time when an aperture of a capsule device according to one embodiment remains closed;

FIG. 4B shows a plot of luminescent intensity during a time when an aperture of a capsule device according to one embodiment has opened;

FIG. 5A shows a perspective view of a threaded capsule of an ingestible capsule device according to one embodiment;

FIG. 5B shows a perspective view of an unthreaded capsule of an ingestible capsule device according to one embodiment;

FIG. 5C shows three substrate layers of an ingestible capsule device according to one embodiment;

FIG. 5D shows a front view of an electronics unit of an ingestible capsule device according to one embodiment;

FIG. 5E shows a top view of an electronics unit of an ingestible capsule device according to one embodiment;

FIG. 6A is a plot of luminescence intensity for various pH values of a fluid sample containing MPO upon exposure to luminol for one embodiment of an ingestible capsule device;

FIG. 6B is a plot of the peak luminescence intensities for the various pH values of FIG. 6A;

FIG. 7A shows an experimental setup for evaluating various concentrations of pH buffers for one embodiment of an ingestible capsule device;

FIG. 7B shows the pH values produced by the concentrations evaluated using the setup of FIG. 7A for one embodiment of an ingestible capsule device;

FIG. 8A shows a heat map diagram of luminescence intensity peak values obtained from the interaction of UHP and luminol in the absence of MPO for one embodiment of an ingestible capsule device;

FIG. 8B shows a heat map diagram of luminescence intensity peak values obtained from the interaction of UHP and luminol in the presence of MPO for one embodiment of an ingestible capsule device;

FIG. 8C shows a heat map diagram of the difference between the intensity peak values of FIG. 8A and the intensity peak values of FIG. 8B for one embodiment of an ingestible capsule device;

FIG. 9A shows time curves of the intensities of luminescence resulting from various concentrations of MPO being exposed to luminol in one embodiment of an ingestible capsule device;

FIG. 9B shows the areas under each time curve of FIG. 9A for one embodiment;

FIG. 9C shows time curves of voltages generated by a photodetector in response to luminescence resulting from various concentrations of MPO being exposed to luminol in one embodiment of an ingestible capsule device;

FIG. 9D shows the areas under each time curve of FIG. 9C for one embodiment;

FIG. 10A shows time curves of voltages generated by a photodetector in response to luminescence resulting from various concentrations of various biomarkers being exposed to luminol in one embodiment of an ingestible capsule device;

FIG. 10B shows the areas under each time curve of FIG. 10A for one embodiment;

FIG. 11A shows an experimental setup for evaluating ex vivo detection of MPO for one embodiment of an ingestible capsule device, wherein the capsule device is prepared for insertion into a porcine small intestine;

FIG. 11B shows the experimental setup of FIG. 11A, wherein the capsule device is inserted into the porcine small intestine;

FIG. 11C shows time curves of voltages generated by a photodetector in response to luminescence resulting from various concentrations of MPO in the experimental setup of FIG. 11A; and

FIG. 11D shows the areas under each time curve of FIG. 11C for one embodiment.

### DETAILED DESCRIPTION

As noted above, conventional methods of detecting and evaluating conditions within a GI tract of a patient may include monitoring symptoms, endoscopies, colonoscopies, and capsule endoscopies. As used herein, the term “condition” is intended to encompass not only disease conditions or disorders such as IBD, but also a general state of the GI tract, including the presence or absence of certain circumstances, qualities, or substances such as enzymes, biomarkers, microbiota, etc.

These conventional methods may be used to detect or evaluate various conditions including IBD or related conditions. However, these methods may be time-consuming, expensive, unpleasant, and/or invasive, and may require a skilled medical practitioner. These factors may pose challenges for patients requiring regular monitoring. For example, the discomfort and pain caused by endoscopic procedures (which frequently involve sedation) may decrease the willingness or ability of a patient to undergo such a procedure. Additionally, the predictive value of these methods may be very limited. Therefore, patients may be required to visit a doctor frequently to re-assess their condition, perhaps as often as every 2-4 months.

It has been appreciated that the level of certain biomarkers may become elevated during flare-ups in patients suffering from IBD, cancer, or other GI conditions. These biomarkers may include myeloperoxidase (MPO), tumor necrosis factor-alpha, interleukin (IL), C-reactive protein (CRP), calprotectin, lactoferrin, and/or others. For example, studies indicate that the biomarker MPO is the primary enzyme that is released by polymorphonuclear leukocytes which accumulate at inflammation sites. Accordingly, a change in the concentration of MPO may be a useful indicator of inflammation or mucosal damage resulting from an occurrence or flare-up of IBD or GI-related cancer.

Based on these discoveries, stool sampling techniques have been developed to detect and evaluate the presence of such biomarkers in a patient's stool. These stool sampling techniques may allow for noninvasive diagnosis or monitoring of IBD (or related conditions), which can be done at a reduced cost and with less disruption to the patient when compared with the traditional methods above. However, while a level of a biomarker in a patient's stool may indicate (to some extent) the presence, absence, or status of a disease condition, the level of the biomarker in the stool may also be highly dependent on a variety of other factors. Exemplary factors that may affect the level of a fecal biomarker can include, for example, the patient's diet, the water content in stool samples, and the disease location. For example, a patient with ileal CD may have massive ulcers. However, the ileal disease location may result only a very low level of fecal biomarkers. Accordingly, these stool sampling methods may provide a simple assessment of IBD, but may be imprecise or unreliable in determining the location or status of a disease condition.

In view of the above, the inventors have recognized and appreciated the benefits of an ingestible capsule device for detecting and/or evaluating conditions within the GI tract. In some embodiments, an ingestible capsule device according to the present disclosure may detect or evaluate the presence of an enzyme or other biomarker at a particular point in the GI tract of a patient. For example, an ingestible capsule device may detect the presence of MPO within the small intestine of a patient to monitor or diagnose IBD or GI-related cancer. This may allow for diagnosis or monitoring that is less invasive, less disruptive, and less expensive than traditional methods such as endoscopic procedures, while providing a higher a degree of reliability and precision than methods such as stool sampling.

Some methods and devices for detecting conditions in the GI tract may include ingestible capsule devices which include fluorescence imaging capabilities. For example, capsule devices with fluorescence imaging capabilities may be used in the diagnosis of certain GI-related cancers or other disease conditions. However, capsule devices which utilize fluorescence imaging may require the inclusion of an excitation light source within the capsule. The excitation light source may require a substantial source of electrical power to operate, may be associated with a high degree of complexity in manufacturing, and may not be reliable.

In view of the above, the inventors have recognized and appreciated the benefits of an ingestible capsule device that uses luminescence or chemiluminescence to detect and evaluate conditions within the GI tract. Capsule devices with luminescence capabilities may detect or evaluate GI conditions using chemical interactions to produce and detect a luminescent light in the presence of certain conditions or compositions of matter. Such devices may operate without the use of an excitation light source, thereby reducing the power requirements and manufacturing complexity in comparison to fluorescence capsule devices.

In some embodiments, an ingestible capsule device may use luminescence or chemiluminescence to detect or evaluate a target enzyme or other biomarker which is indicative of a disease condition. For example, an ingestible capsule device may include a luminescent substrate configured to emit a luminescent light upon exposure to GI fluid containing a luminescing trigger. The luminescing trigger may be the target enzyme or biomarker, or the luminescing trigger may be another chemical derived from the target enzyme or biomarker. In some embodiments, the capsule may include a photodetector configured to detect the presence, absence, or intensity of luminescent light emitted by the luminescent substrate.

In some embodiments, a target biomarker may include myeloperoxidase (MPO), tumor necrosis factor-alpha, interleukin (IL), C-reactive protein (CRP), calprotectin, lactoferrin, or others. In some such embodiments, a luminescent substrate of a capsule device may be infused with a solution containing a luminescing agent such as cypridina luciferin, firefly luciferin, oxalate, lucigenin, luminol (C₈H₇N₃O₂), a derivative of luminol, and/or other chemiluminescent molecule(s). In some embodiments, the luminescing agent may be used in combination with quantum dots or nanoparticles configured to emit additional light when exposed to a luminescence emitted by the luminescing agent. In embodiments which use luminol, a luminescing trigger may be an oxidizing agent, such as hypochlorous acid (HOCl). In some such embodiments, an active molecule may be included to interact with the target biomarker in order to produce the luminescing trigger. For example, in embodiments which use luminol and wherein the target biomarker is MPO, the capsule device may include urea hydrogen peroxide (UHP). The UHP may interact with the MPO to produce the HOCl required to interact with luminol. The interaction between HOCl and luminol may generate a luminescent light which indicates the presence, absence, or concentration of MPO in a GI fluid sample.

The capsule device may further be configured to transmit a wireless signal. The wireless signal may relay information about a detection signal generated by the photodetector of the capsule device to a separate receiving device external to the patient. In some embodiments, the wireless signal may indicate the presence, absence, or intensity of a luminescent light detected at a particular location within the patient's GI tract, or at a time which corresponds to the particular location. The presence, absence, or intensity of the luminescence may indicate the presence, absence, or concentration of the biomarker, which in turn may indicate the status of a disease or other GI condition.

In some embodiments, an ingestible capsule device of the present disclosure may target a particular region of the GI tract (e.g., for monitoring or evaluation). For example, the capsule device may include a capsule having a sampling aperture which allows GI fluid to enter the capsule for evaluation. In some embodiments, the sampling aperture may be closed with one or more layers of a biodegradable or enteric coating. The enteric coating may be selected to degrade at a desired pH level, allowing the sampling aperture to remain closed until the enteric coating is exposed to the desired pH level. Because the pH level of the GI tract varies along the length of the GI tract, the ingestible capsule device may be designed to target a particular region of the GI tract by selecting or configuring the enteric coating to degrade at a particular pH level. Multiple layers of enteric coatings may also be used to target specific regions of the GI tract, such as the colon. When multiple layers of enteric coatings are used, each layer of enteric coating may be configured to degrade at different pH levels, thus allowing more specific targeting of regions of the GI tract. Suitable coating materials may include, but are not limited to, pH-sensitive polymeric materials such as basic butylated methacrylate (EUDRAGIT EPO), poly methacrylic acid-co-ethyl acrylate (EUDRAGIT L 100-55), poly methacrylic acid-co-methyl methacrylate (EUDRAGIT L100), hydroxypropyl methylcellulose phthalate (HP-55), hypromellose phthalate (HPMCP), cellulose acetate phthalate (CAP), and polyvinyl acetate phthalate (PVAP). Exemplary ingestible sampling capsules that employ enteric coatings with multiple layers that degrade at different pH levels are described in further detail in U.S. Provisional App. No. 63/320,825, filed on Mar. 17, 2022, which is incorporated for all purposes herein in its entirety. Similar capsules and/or multi-layer enteric coatings may be employed with any of the embodiments described herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

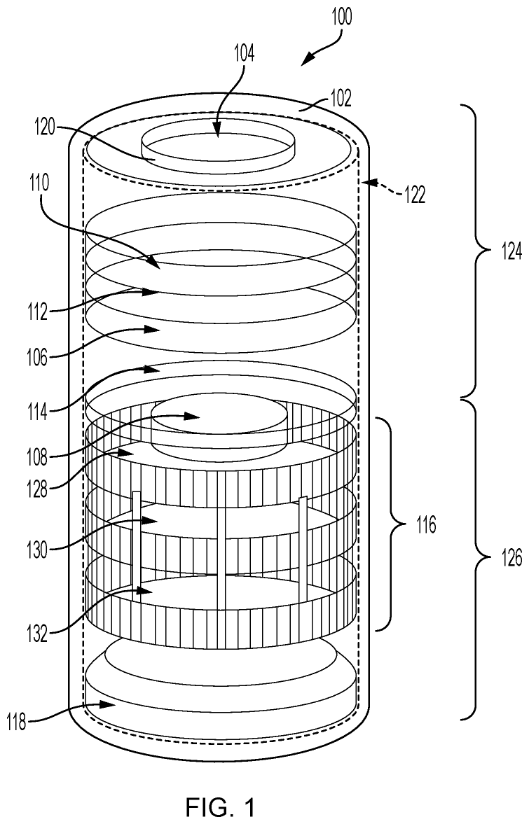

FIG. 1 depicts one embodiment of an ingestible capsule device according to the present disclosure. In the embodiment shown, a device 100 may include a capsule 102 having an internal cavity 122. The capsule 102 may also include a sampling aperture 120 to allow fluid exchange between the cavity 122 and a surrounding environment of the device 100. The aperture 120 may be filled, covered, or otherwise closed by an aperture closure 104. The aperture closure 104 may comprise a biodegradable material, such as an enteric coating. The biodegradable material may be selected or configured to degrade at a desired point along the GI tract of the patient. For example, an enteric coating may be selected to degrade at a pH level corresponding to a pH level of the small intestine such that the device 100 may be configured to evaluate a sample from the small intestine.

The device 100 may include one or more layers of a substrate material, such as two layers, three layers, four layers, etc. In the embodiment shown, the device 100 may include a luminescent substrate 106, a first additional substrate layer 110, and a second additional substrate layer 112, although other embodiments may include more substrate layers or fewer substrate layers. A substrate material may comprise any appropriate material for carrying an active molecule therein, such as paper, fabric, polymeric materials (including polymeric mesh, polymeric membranes, and others), synthetic materials, composite materials, hydrogels, freeze-dried hydrogel matrices, or others. In some embodiments, the substrate material may comprise filter paper or cellulose paper. Each layer of substrate material may include one or more active molecules. Each active molecule may be selected to produce a desired chemical interaction with a sample of GI fluid in the capsule device, or with a component or constituent of the GI fluid sample.

The luminescent substrate 106 may include a luminescing agent. The luminescing agent may be an active molecule which produces a luminescent light when exposed to a luminescing trigger molecule. For example, in some embodiments, the luminescing agent may be luminol. In other embodiments, the luminescing agent may be a derivative of luminol, or the luminescing agent may be cypridina luciferin, firefly luciferin, oxalate, lucigenin, and/or other chemiluminescent molecule(s). In some embodiments, the luminescing agent may be used in combination with quantum dots or nanoparticles configured to emit additional light when exposed to a luminescence emitted by the luminescing agent. The inclusion of quantum dots or nanoparticles may increase the amount of light generated in response to a given concentration of the luminescing trigger, thereby increasing a sensitivity of the capsule device.

The luminescing trigger molecule may be indicative of a condition within the GI tract. In some embodiments, the luminescing trigger may be an enzyme, biomarker, biomolecule, or other indicator of a GI condition. In other embodiments, the luminescing trigger may be an active molecule derived from an enzyme, biomarker, biomolecule, or other indicator of a GI condition. For example, in embodiments which use luminol as the luminescing agent, a luminescing trigger may be an oxidizing agent such as hypochlorous acid (HOCl). The device 100 may derive the oxidizing agent or other luminescing trigger from an enzyme or biomarker through chemical interaction between a GI fluid sample and one or more active molecules in one or more substrate layers of the capsule device. In the embodiment shown, a luminescing trigger may be derived through chemical interaction between a GI fluid sample and one or more active molecules infused in a first additional substrate layer 110 and/or a second additional substrate layer 112.

For example, in the embodiment shown, for a device 100 configured to detect MPO in a GI fluid sample, the second additional substrate layer may be infused with a solution having an appropriate concentration of UHP, such that the interaction between the UHP and the MPO may produce HOCl. The HOCl may act as an oxidizing agent or luminescing agent to interact with the luminol of the luminescent substrate 106 to produce a luminescent light to indicate the presence, absence, or concentration of MPO in the GI fluid sample.

Additionally or alternatively, additional substrate layers may be included and configured to produce effects other than deriving the luminescing trigger. In some embodiments, an active molecule in an additional substrate layer may be provided to adjust or modify a property of a GI fluid sample. For example, in some embodiments, an additional substrate layer may include a pH buffer agent. A pH buffer agent may be an active molecule which is capable of adjusting a pH level the GI fluid sample. A pH buffer agent may include 4-(Cyclohexylamino)-1-butanesulfonic acid (CABS) or a similar active molecule. In the embodiment shown, the first substrate layer 110 may be infused with a solution having an appropriate concentration of a pH buffer agent such as CABS. As will be described in greater detail in the Examples section below, it may be desirable to adjust a pH level or other property of a GI fluid sample in order to optimize a luminescent intensity produced by the luminescing substrate 106.

In some embodiments, the device 100 may include a translucent partition 114. The translucent partition 114 may separate the cavity 122 into a first portion 124 and a second portion 126 of the cavity 122. Further, the translucent partition 114 may form a liquid-tight seal between the first portion and the second portion of the cavity while permitting light to pass between the first and second portions. In various embodiments, the translucent partition may comprise glass, plastic, or any other appropriate material for forming a seal within a capsule while permitting light to pass therethrough.

According to some embodiments, an ingestible capsule device may include an electronics unit 116. Some embodiments may further include a power supply 118. In some embodiments, the electronics unit 116 may include a sensor interface module 128, a signal processing module 130, and a data collection and transmission module 132. The sensor interface module 128 may include a photodetector 108 for detecting the presence, absence, or intensity of a luminescent light. In some embodiments, the photodetector 108 may comprise a photodiode, such as a single-photon avalanche diode (SPAD). In other embodiments, the photodetector may comprise a microplate reader or any other appropriate type of photodetector.

The signal processing module 130 may be in communication with the sensor interface module 128 and/or the photodetector 108 to receive and process a detection signal from the photodetector 108. In some embodiments, the signal processing module 130 may be configured to control a noise level of the detection signal or to otherwise process the detection signal.

The data collection and transmission module 132 may be in communication with the signal processing module 130. The data collection and transmission module 132 may be configured to transmit a wireless signal containing information that is based at least in part on information from the detection signal. The data collection and transmission module 132 may be configured to transmit the wireless signal via any appropriate communication protocol, including radio frequency (RF) protocols, WiFi protocols, Bluetooth, long range (LoRa) networking protocols, multicast wireless sensor networks (e.g., ANT), intra-body communication networks, and/or others.

The power supply 118 may be included to provide an appropriate level of electrical power to the various components of the electronics unit 116. The power supply 118 may comprise a battery or any other appropriate source of electrical power.

An exemplary embodiment of an electronics unit 116 in accordance with the present disclosure is described in greater detail in the Examples section below. However, it will be appreciated that an electronics unit may include any appropriate component or components for detecting the presence, absence, or intensity of a luminescent light and relaying information about the light detected. In this respect, the disclosure is not limited to the specific components described below.

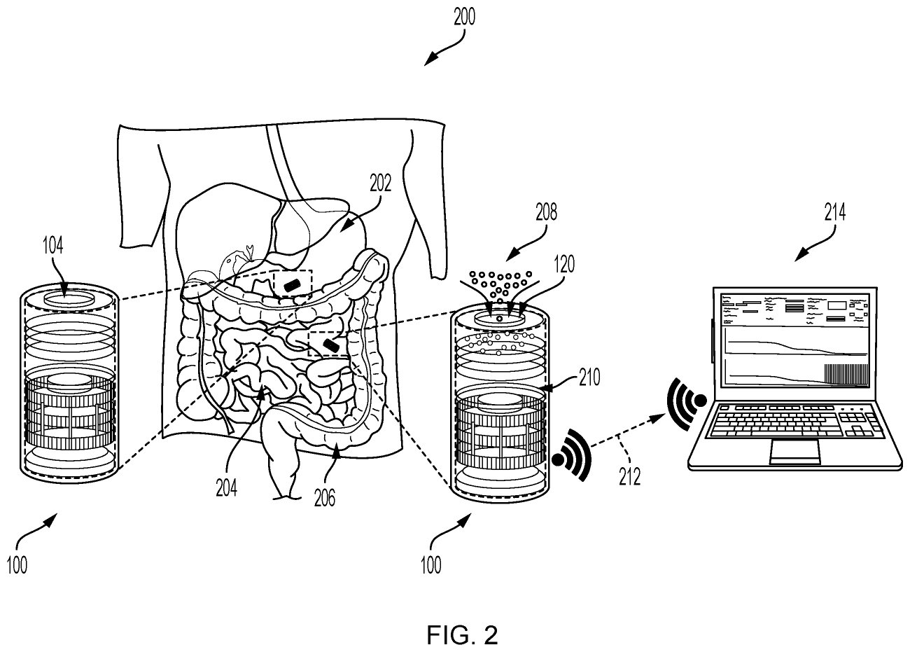

The operation of an ingestible capsule device such as the one shown in FIG. 1, will now be described with reference to FIG. 2. A device 100 may be ingested by a patient 200. While the device 100 is in the stomach 202 of the patient 200, the sampling aperture 120 may remain closed by the aperture closure 104. In the embodiment shown, the aperture closure 104 may comprise a biodegradable or enteric material configured to degrade at a pH level corresponding to a portion of the small intestine 204. Accordingly, when the device 100 reaches the small intestine 204, the aperture closure 104 may degrade or dissolve, thereby allowing a sample of GI fluid to pass through the aperture 120 and into the cavity 122 of the device 100. It will be appreciated that in other embodiments, the aperture closure 104 may be configured to degrade or dissolve at another location of the GI tract, such as the large intestine 206.

The sample of GI fluid passing through the aperture 120 may contain an enzyme or other biomarker 208 indicative of a GI condition to be monitored or evaluated. The enzyme or biomarker 208 may be a luminescing trigger, or a luminescing trigger may be chemically derived from the enzyme or biomarker 208 as described herein. When a luminescent substrate of the device 100 is exposed to the luminescing trigger, the luminescent substrate may emit a luminescent light 210. The luminescent light 210 may be detected by a photodetector 108 of the device 100, as shown in FIG. 1 above. The photodetector may generate a detection signal as described above, the detection signal indicating the presence, absence, or intensity of the luminescent light 210.

The capsule device 100 may transmit a wireless signal 212 to an external receiving device 214. The receiving device 214 may be any device capable of receiving the wireless signal 212 from the capsule device. The receiving device may be monitored by a user or a medical professional. Alternatively or additionally, the receiving device 214 may be configured to store information from the wireless signal 212 for subsequent analysis.

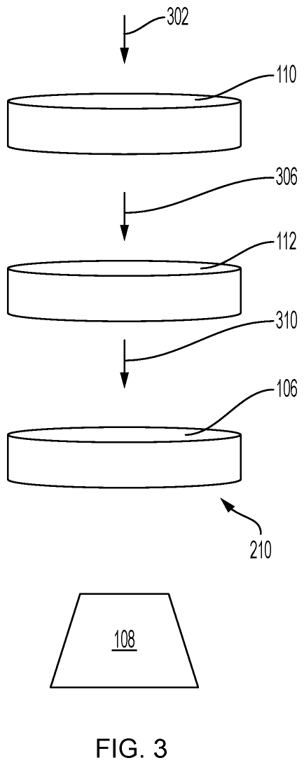

One example of a mode of operation for an embodiment of a capsule device according to the present disclosure will now be described with reference to FIG. 3. In this example, the device may be configured to monitor or evaluate an IBD condition by detecting MPO in a GI fluid sample. This exemplary embodiment may include luminol as a luminescing agent within a luminescent substrate 106. In this embodiment, interaction between MPO and luminol may produce insufficient luminescent light to allow for useful analysis or evaluation. Accordingly, a capsule device of this embodiment may derive a luminescing trigger from MPO through chemical interaction with an active molecule. The luminescing trigger in this embodiment may be HOCl. The luminescing trigger may be derived through chemical interaction between the MPO of the GI fluid sample and an active molecule infused into an additional substrate layer. The active molecule in this particular embodiment may be urea hydrogen peroxide (UHP). The UHP may interact with MPO to produce HOCl. The HOCl may interact with the luminol of the luminescing substrate to produce a luminescent light.

Additionally, as will be described further in the Examples section below, a pH value of the GI fluid sample may affect the intensity of the luminescent light produced by the interaction of HOCl and luminol. Accordingly, it may be desirable to adjust the pH value of the GI fluid sample. In the particular embodiment described here, the pH value may be adjusted to a desired value using a CABS-infused substrate layer.

While this example is intended to illustrate the operating principles of an ingestible capsule device, it will be appreciated that these principles may be applied and adapted to produce an ingestible capsule device for any appropriate application, including the monitoring or evaluation of other GI conditions through the use of other active molecules and/or other electronic components to detect other target biomarkers as appropriate for a given application. As a further example, a different number and/or arrangement of substrates (e.g., substrates infused with a different chemical or combination of chemicals) can be used in accordance with the techniques described herein. Accordingly, the disclosure is not limited to the examples described herein that are targeted specifically to detect MPO using luminol, UHP, and the CABS pH buffer.

At a first stage 302, a sample of GI fluid may contain a variety of constituent components, including a target enzyme or other biomarker to be detected. In some embodiments, the target biomarker may be MPO. In other embodiments, the target biomarker may be tumor necrosis factor-alpha, interleukin (IL), C-reactive protein (CRP), calprotectin, lactoferrin, or any appropriate biomarker.

A first substrate layer 110 may be configured to chemically interact with the sample of GI fluid. As described above, the first substrate layer 110 may be configured to adjust a pH of the GI fluid. This pH adjustment may be desirable in order to optimize the sample for luminescence. In some embodiments, the first substrate layer 110 may be a layer of filter paper infused with a first buffer solution containing an appropriate concentration of CABS. In other embodiments, the first substrate layer 110 may include other active molecules, including other pH buffers or active molecules which adjust fluid properties other than pH, such as salinity, viscosity, electrical conductivity, and/or others. For example, in some embodiments, a substrate layer may be infused with a redox buffer solution or redox buffer molecule in order to control an oxidation/reduction potential of the GI fluid sample or a constituent thereof. In some embodiments, a substrate layer may be infused with a detergent or other compound to control a viscosity of the GI fluid sample.

According to the presently-described embodiment, a pH of the GI fluid sample may be different at a second stage 306 than a pH of the GI fluid sample at the first stage 302. For example, in some embodiments, a concentration of CABS solution infused in the first substrate layer may be selected to raise the pH of the GI fluid sample to a pH value of 11. In other embodiments, other buffer solutions may be used in appropriate concentrations to adjust the pH to any desired level, as the disclosure is not limited in this regard.

Accordingly, in some embodiments, a concentration of pH buffer solution may be selected to obtain a pH value that may be greater than or equal to 0, 5, 7, 10, and/or any other appropriate pH value. Additionally, the concentration of pH buffer solution may be selected to obtain a pH value that may be less than or equal to 14, 12, 11, 10, and/or any other appropriate pH value. Combinations of the foregoing are contemplated including, for example, a pH value of greater than or equal to 0 and less than or equal to 14, greater than or equal to 10 and less than or equal to 12, and/or any other appropriate combination of the foregoing. Of course, while particular ranges for the desired pH value are provided above, it should be understood that other ranges both greater than and less than those noted above are also contemplated as the disclosure is not limited in this fashion.

Additionally, in some embodiments where CABS is used as a pH buffer to adjust a pH value of a GI sample containing MPO for detection by interaction with luminol, a concentration of the CABS buffer solution selected to obtain the desired pH value may be greater than or equal to 0.2 M, 0.4 M, 0.6 M, 0.8 M and/or any other appropriate molarity or concentration. Additionally, the concentration of the CABS buffer solution may be less than or equal to 1.4 M, 1.2 M, 1.0 M, 0.8 M, and/or any other appropriate molarity or concentration. Combinations of the foregoing are contemplated including, for example, a concentration of greater than or equal to 0.2 M and less than or equal to 1.4 M, greater than or equal to 0.8 M and less than or equal to 1.0 M, and/or any other appropriate combination of the foregoing. Of course, while particular ranges for the concentration of CABS buffer solution are provided above, it should be understood that other ranges both greater than and less than those noted above are also contemplated as the disclosure is not limited in this fashion.

A second substrate layer 112 may be configured to chemically interact with the GI fluid sample from the second stage 306. In some embodiments, the second substrate layer 112 may be configured to interact with the GI fluid in order to produce a luminescing trigger. For example, in some embodiments the second substrate layer 112 may be a layer of filter paper infused with a solution containing an appropriate concentration of urea hydrogen peroxide (UHP). In some embodiments, a volumetric concentration of UHP solution selected to obtain HOCl from MPO for interaction with luminol may be greater than or equal to 0%, 0.25%, 0.5%, 1.0%, 1.5% and/or any other appropriate concentration. Additionally, the concentration of the UHP solution may be less than or equal to 3.0%, 2.5%, 2.0%, 1.5%, 1.0%, and/or any other appropriate concentration. Combinations of the foregoing are contemplated including, for example, a concentration of greater than or equal to 0% and less than or equal to 3.0%, greater than or equal to 1.5% and less than or equal to 2.5%, and/or any other appropriate combination of the foregoing. Of course, while particular ranges for the concentration of UHP solution are provided above, it should be understood that other ranges both greater than and less than those noted above are also contemplated as the disclosure is not limited in this fashion.

In some embodiments, the UHP may react with the MPO of the GI fluid to produce hypochlorous acid (HOCl). Accordingly, the GI fluid sample at a third stage 310 may include the luminescing trigger. In this example, the GI fluid sample at the third stage 310 may include HOCl.

A luminescing substrate 106 may be configured to emit a luminescent light 210 when exposed to the luminescing trigger. In some embodiments, the luminescing substrate may be infused with a solution containing an appropriate concentration of a luminescing agent. The luminescing agent may be a chemiluminescing agent such as luminol (C₈H₇N₃O₂). In this example, the luminol may interact with the HOCl derived from the MPO as described above to produce the luminescent light 210.

In some embodiments, a concentration of the luminol solution selected to optimize the luminescent intensity in the presence of MPO may be greater than or equal to 1 mM, 10 mM, 15 mM, 20 mM and/or any other appropriate molarity or concentration. Additionally, the concentration of the luminol solution may be less than or equal to 30 mM, 25 mM, 20 mM, 15 mM, and/or any other appropriate molarity or concentration. Combinations of the foregoing are contemplated including, for example, a concentration of greater than or equal to 1 mM and less than or equal to 30 mM, greater than or equal to 20 mM and less than or equal to 30 mM, and/or any other appropriate combination of the foregoing. Of course, while particular ranges for the concentration of luminol solution are provided above, it should be understood that other ranges both greater than and less than those noted above are also contemplated as the disclosure is not limited in this fashion.

It will be appreciated that although the combination of MPO, UHP, and luminol are described herein as being included in one embodiment of the present disclosure, other embodiments may utilize other luminescing triggers, luminescing agents, and/or buffer solutions as the disclosure is not limited in this regard. Additionally, the luminescing agent, luminescing trigger, buffer solutions, active molecules, as well as the combinations and concentrations thereof may be selected and optimized for the detection of any appropriate enzyme or biomarker, including tumor necrosis factor-alpha, interleukin (IL), C-reactive protein (CRP), calprotectin, lactoferrin, and others.

Furthermore, it will be appreciated that while the embodiment of FIG. 3 includes one luminescing substrate layer and two additional substrate layers, other embodiments may include any appropriate number of additional substrate layers. For example, it will be appreciated that in some embodiments, no additional substrate layers will be needed to produce the luminescent light which indicates the presence of a desired enzyme or biomarker. In other embodiments, more than two additional substrate layers may be desirable. Accordingly, the present disclosure is not limited to any particular number of substrate layers.

The luminescent light 210 may be detected by a photodetector 108. In some embodiments, the photodetector 108 may be sufficiently sensitive to detect only trace amounts of the luminescent light 210. For example, in some embodiments the photodetector 108 may be a single-photon avalanche diode. Upon detecting the luminescent light 210, the photodetector 108 may produce a detection signal indicating the presence, absence, or intensity of the luminescent light 210. The detection signal may therefore indicate the presence, absence, or concentration of a luminescing trigger, or the presence, absence, or concentration of the enzyme or biomarker. The detection signal may be processed and transmitted by an electronics unit of the capsule device as described herein.

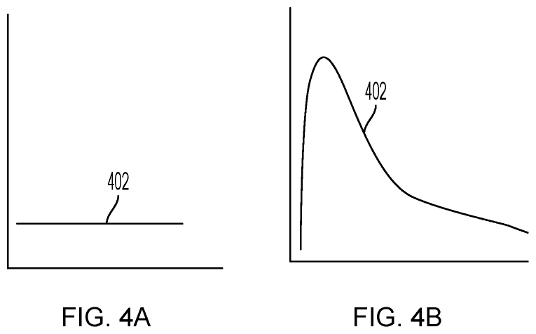

FIG. 4A depicts a luminescent intensity within some embodiments of a device as described herein during a time when the capsule aperture remains closed. For example, the luminescent intensity shown in FIG. 4A may correspond to a time when a device 100 is located in the stomach 202 of a patient 200 as shown in FIG. 2. FIG. 4B depicts a luminescent intensity within some embodiments of a device as described herein during a time when the capsule aperture has opened and a GI fluid sample containing an enzyme or biomarker to be detected has entered the capsule. For example, the luminescent intensity shown in FIG. 4B may correspond to a time when a device 100 is located in the small intestine 204 of a patient 200 as shown in FIG. 2. The increase in luminescent intensity shown in FIG. 4B may be sufficient to generate a detection signal in a photodetector of the device. In some embodiments, the detection signal, or information derived therefrom, may be processed and transmitted wirelessly to a receiving device as described with respect to FIG. 2 above.

### EXAMPLES

### Design and Use-Case for an Experimental Embodiment

One example of an experimental capsule device according to the present disclosure comprised six elements: a capsule, a pH-sensitive biodegradable enteric coating, a stack of three thick filter papers, a glass coverslip, an electronic unit with a photodetector on top, and a 3-volt lithium battery. The capsule utilized a two-part threaded design to facilitate assembly of the capsule device. The pH-sensitive polymeric coating was used to survive the low-pH gastric environment, where the experimental capsule device was intended to remain intact.

This example was intended to evaluate the small intestine. Upon exposure to the targeted inflamed tissue in the small intestine (which has an average pH of 6.8), the enteric coating would dissolve to permit the inflow of MPO (released by neutrophils at the site of inflammation) into the capsule. Subsequently, the three layers of filter paper would start absorbing the MPO and play their role as follows: first, the CABS-infused filter paper would adjust the pH of the incoming sample to a pH of 11; next, the UHP-infused filter paper would interact with the MPO (having pH 11) and produce hypochlorous acid (HOCl); and then, the luminol-infused filter paper would interact with the HOCl and start emitting blue luminescence. At this point, the photodetector would detect the luminescence and convert the photon energy to an electrical signal which would then be transmitted to a receiving device through a wireless transmitter of the electronics unit of the capsule device.

### Exemplary Optimization of Analytical Parameters

The character and intensity of luminescence emitted by the oxidation reaction of luminol may depend on several parameters, including the pH levels and the concentrations of reactants. Therefore, a series of experiments were executed to optimize these parameters around the specific use-case in which luminol is used for optical detection of MPO.

### Optimization of pH

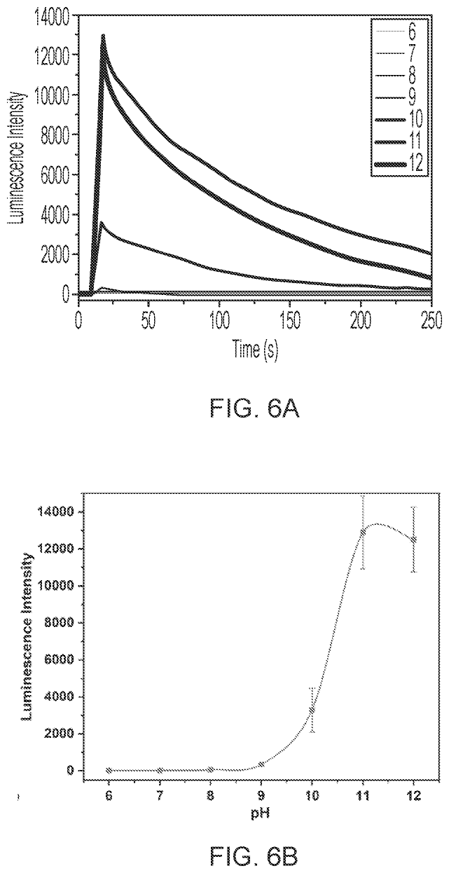

First, the influence of pH on the luminescence intensity was investigated by exposing the MPO to buffers of varying pH, including pH of 6, 7, 8, 9, 10, 11, and 12. For this, a 96-well black microtiter plate was filled with supernatant GI fluid having pH values ranging from 6 to 12. Into each well, 50 μL of UHP (250 μM) was added. Ten seconds after the UHP was added, a fixed volume of luminol was added to each well. Two seconds after the luminol was added (i.e., twelve seconds after the UHP was added), a fixed volume of MPO was added to each well. The mixtures were left to incubate for ten seconds. Following the incubation period, the luminescence spectra of each reaction was recorded for 250 seconds using a BMG microplate reader. The results for each pH level are shown in FIGS. 6A and 6B. As can be seen from FIGS. 6A and 6B, it was observed that the maximum luminescence intensity was produced using a pH value of 11.

However, pH in the small intestine region varies from 6.2 to 7.4. As can be seen from FIGS. 6A and 6B, the luminescence intensity observed for that range of pH values was significantly less than the intensity at the pH value of 11. Therefore, the inventors recognized and appreciated the benefits of adjusting the pH value of a GI fluid sample in order to optimize the luminescence intensity in the disclosed capsule device. In the experimental embodiment disclosed here, a CABS-infused filter paper was included in the capsule in order to adjust the pH value of a GI fluid sample.

The inclusion of the CABS-infused filter paper necessitated further optimization to determine an appropriate concentration of CABS within the filter paper for obtaining the desired pH value of 11 from a GI fluid sample having an initial pH value between 6 and 7. Several dilutions (0.2M, 0.4M, 0.6M, 0.8M, 1.0M, 1.2M and 1.4M dilutions) of CABS stock solutions were prepared. A fixed volume (100 μL) of each dilution was used to drop cast onto circular discs of thick filter paper. The resulting filter papers (having different concentrations of CABS) were left overnight to dry at room temperature.

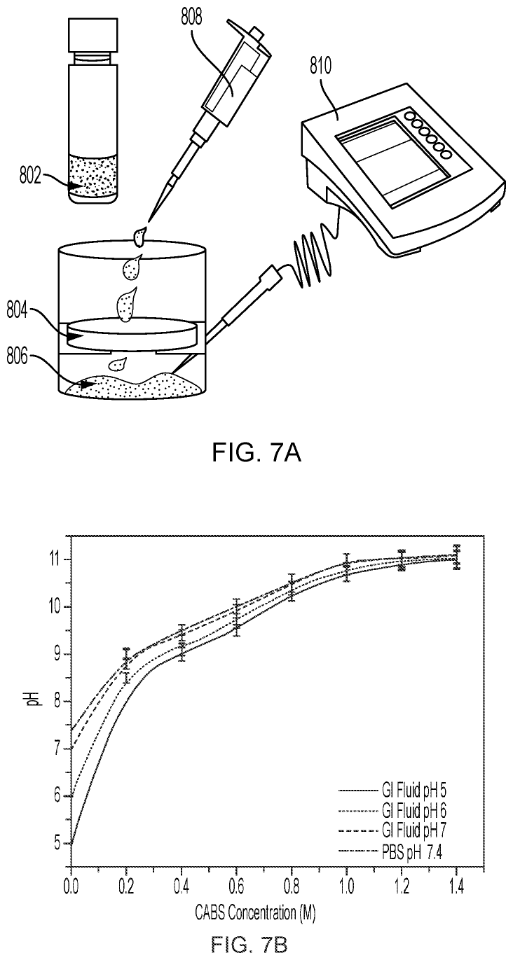

These CABS-infused dried filter papers were used to determine which dilution of CABS stock solution best achieved conversion of GI fluid from in vivo pH level of around 6-7 to the desired pH level of around 11. Three different GI fluid samples having pH levels of 5, 6, and 7, as well as a phosphate-buffered saline (PBS) buffer solution having a pH of 7.4 were used as the testing samples. FIG. 7A shows a diagram of the experimental setup for the pH conversion experiment described above, and FIG. 7B shows the obtained results. In FIG. 7A, the GI fluid samples 802 or PBS buffer solution of various pH values was pipetted onto and filtered through the filter papers 804 infused with the various dilutions of CABS stock solutions. A pH meter 810 was used to measure the final pH of the filtered GI fluid 806.

The outcomes shown in FIG. 7B indicated that the filter paper infused with 1.0M CABS solution was sufficient to convert the pH of each to around the desired pH value of 11. Therefore, 1.0M CABS solution was selected as an appropriate concentration of CABS within the filter paper for obtaining the desired pH value of 11 from a GI fluid sample having an initial pH value between 6 and 7. Although the particular methods, parameters, and goals of these experiments resulted in the selection of these particular compounds, values, and concentrations, it will be appreciated that deviation from these particular methods, parameters, and goals for other applications may result in the selection of any appropriate compound, value, or concentration for any of the variables discussed herein.

### Optimization of the Ratio of UHP and Luminol Concentrations

The ratio of UHP and luminol concentration also affects the oxidation reaction of the presently-described embodiment, and thus the chemiluminescence intensity. Therefore, various concentrations of UHP were evaluated with various concentrations of luminol. It will be appreciated that, in the in vivo context, not all GI fluid will contain significant amounts of MPO. However, UHP and luminol may interact during use of a capsule device regardless of whether the GI fluid contains MPO. Therefore, the various ratios of UHP and luminol were evaluated both in the presence of MPO and in the absence of MPO. Because the capsule device of the presently-described embodiment was intended to detect MPO by evaluating the intensity of luminescence generated by the introduction of MPO into the capsule, the difference between luminescence in the absence of MPO and in the presence of MPO (rather than solely the intensity of luminescence in the presence of MPO) was evaluated as being indicative of the introduction of MPO.

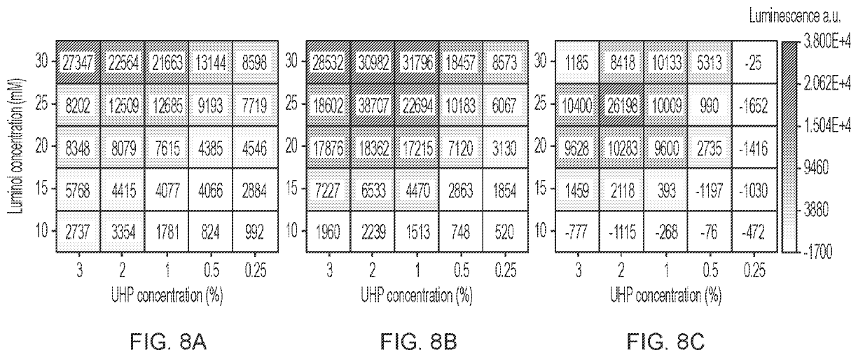

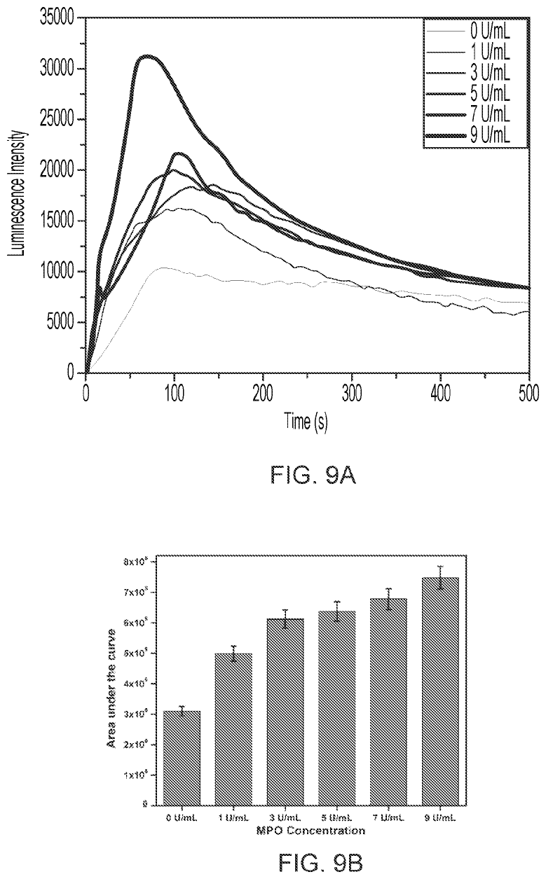

FIG. 8A shows a heat map diagram of luminescence intensity peak values obtained from the interaction of UHP (horizontal axis) and luminol (vertical axis) in the absence of MPO with water as a control. It was observed that the luminescence intensity peak effectively increased with an increasing concentration of both UHP and luminol. This may be due to, for example, the strong oxidation reaction caused by excess amounts of both reactants.

FIG. 8B shows a heat map diagram of luminescence intensity peak values obtained from the interaction of UHP and luminol in the presence of MPO (at a concentration of 7 U/mL). As noted above, the effect of introducing MPO to a given ratio of UHP and luminol was evaluated by comparing the luminescence intensities peak values both in the presence and in the absence of MPO for the given ratio.

Accordingly, FIG. 8C shows a heat map diagram of the difference between the intensity peak values of FIG. 8A and the intensity peak values of FIG. 8B. As shown in FIG. 8C, the most significant increase in the luminescence intensity peak value occurred with 2% UHP and 25 mM luminol concentration. Therefore, 2% UHP and 25 mM luminol concentration were selected for further analytical investigations.

### Exemplary Device Fabrication

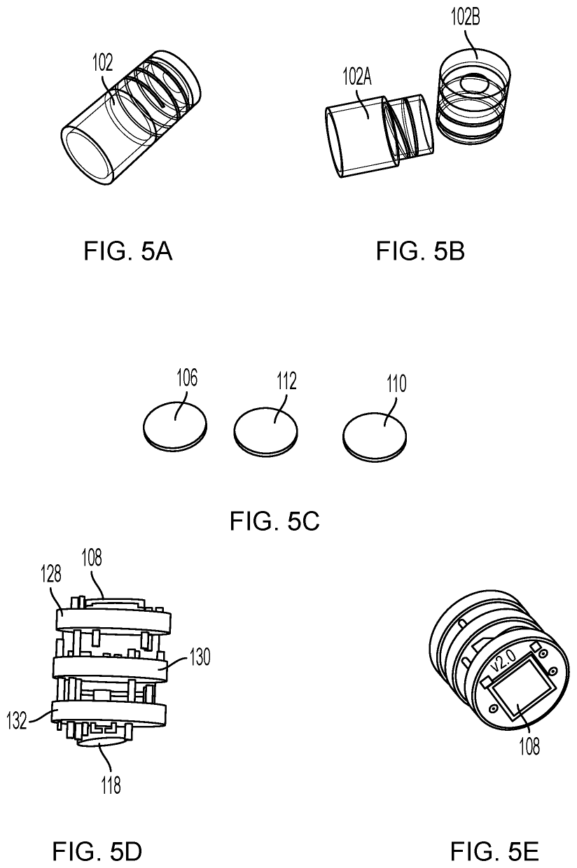

As shown in FIGS. 5A-5B, in one example, a capsule 102 for an ingestible device was manufactured using 3D printing. The capsule included two portions: a body portion 102A and a cap portion 102B. The capsule was designed using SolidWorks (Dassault Systems). The design was 3D printed using a biocompatible resin (EN-ISO 10993-1:2009/AC: 2010, USP Class VI) obtained from Formlabs, Inc., using a stereolithographic (SLA) printing process on a Form 2 printer from Formlabs, Inc. The SLA process used a layer thickness of 50 μm. After printing was complete, the capsules were washed in 99% isopropyl alcohol (IPA) for 15 minutes, and cured using an ultraviolet (UV) photocuring device (from Formlabs, Inc.) for another 60 minutes to complete polymerization of the resin.

After that, a computer-controlled CO₂ laser was used to cut a 6-mm diameter sampling aperture 120 in the cap portion 102B. The laser was a PLS6MW cutting and engraving system from Universal Laser, Inc., Scottsdale, AZ, and was set to an operating wavelength of 10.6 μm. Subsequently, the sampling aperture 120 was filled with a pH-sensitive coating and allowed to cure at room temperature overnight.

As shown in FIG. 5C, a large sheet of filter paper was cut into small circular discs, each disc having a diameter of 5 mm. Active molecules (CABS, UHP, and luminol) were infused into different filter paper discs and the discs were stacked such that the UHP-infused paper 112 was sandwiched between the CABS-infused paper 110 on one side and the luminol-infused paper on the opposite side. The stack of discs was placed in the cap portion 102B of the capsule 102 such that the CABS-infused paper was nearest to the sampling aperture 120.

### Electronics Unit

An electronics unit was included to measure luminescent light output with maximum sensitivity and to provide continuous wireless luminescence measurements. As shown in FIGS. 5C and 5D above, the electronics unit 116 comprised three distinct and modular systems, combined into a single unit. The unit included a sensor interface module 128, an analog signal processing module 130, and a data collection and transmission module 132. Three modular custom printed circuit boards (PCB) were designed to house the electronics. Each of the three modules was contained in a single, 8-mm diameter, circular PCB. The three boards were stacked onto each other with the sensor interface module on one end, signal processing module in the middle, and data collection and transmission module at the other end.

The sensor interface module 128 was provided to interact with the luminescent chemistry directly and to convert light intensity into a representative current. To achieve a wide functional range, a single-photon avalanche diode (SPAD) array from Onsemi (MicroFC-30035-SMT) was used as the photodetector 108 to measure light produced from the MPO reaction. The sensor was biased between −25 V and −30 V with an inverting DC-DC converter from Analog Devices (LT3462). Adjustment of this bias voltage may serve as a primary method for tuning the sensitivity of the device. To allow for extended battery life when the reaction is not expected to occur, the sensor interface was capable of being shut down to draw near-zero current by switching an enable pin on the DC-DC converter.

The analog signal processing module 130 provided noise rejection and amplification to isolate measurements from the sensor interface and convert them to usable voltages. A transimpedance amplifier (TIA) was included to convert SPAD currents to voltages between 0 and 1.8 V. The TIA gain was used to balance the overall gain when the SPAD bias voltage was adjusted. This limited the output of sample signals between 0 and 1.8 V, utilizing the entire range of the ADC. Example schematics of the bias voltage circuit and TIA can be found in the DC-DC converter's and SPAD's datasheets.

Finally, in the data collection and transmission module 132, a microcontroller (nRF52832, ARM M4 processor) collected analog measurements and sent representative digital signals wirelessly to an external receiving device (nRF51822, ARM M0 processor). The nRF52832 controlled all electronic components in the capsule with 1.8 V digital logic and converted the TIA output voltage to a digital signal with its onboard digital-to-analog converter. The nRF52832 and nRF51822 microcontrollers provided built-in RF communication protocols that were used to transmit data collected from inside the GI tract to an external system. In this example, a Raspberry Pi and the nRF51822 were integrated into a single “base station” receiving device to forward collected data to a WiFi source that was accessible by any laptop or other computing device with WiFi capabilities.

Capsule active and shutdown current draw were recorded to estimate battery life. The device was powered at 3.1 V and input current was 17.5 mA active and 3.1 mA shutdown, recorded with an Agilent 34401A digital multimeter. Packaged with two 1.55 V, 23 mAh silver-oxide batteries in series (to produce 3.1 V), the capsule electronics system was configured to actively record light for over an hour, or to sleep for over 7 hours. It was configured to continue transmitting data 10 meters away from a base station through air and to fit within a standard 000 capsule.

### MPO Detection Performance

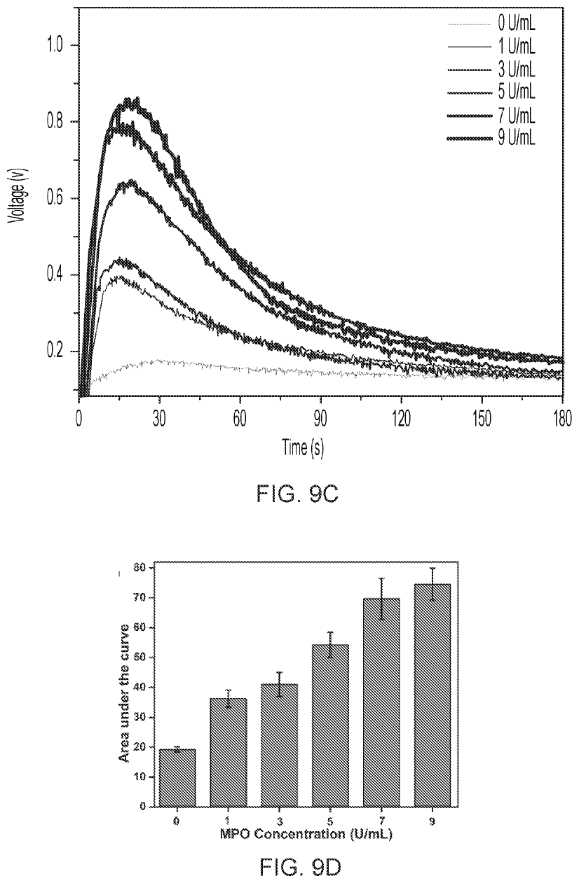

Initially, the analytical performance of the sensor interface module of the electronics unit was evaluated by recording the luminescence spectra generated through different concentrations of MPO. This was done using a conventional BMG Clariostar microplate reader. FIG. 9A shows the luminescence spectra of the oxidation reaction at an optimal wavelength of 425 nm with various concentrations of MPO. As will be appreciated from FIG. 9A, the luminescence intensity increased gradually with an increase in MPO concentration (0-9 U/mL) without significant alteration in the shape of luminescence spectra. This intensification in the luminescence with increased concentration of MPO may be due to the formation of more oxidant (HOCl) through the reaction of MPO and UHP. The increase in HOCl may further oxidize the luminol and thus produce the blue color luminescence. FIG. 9B shows a plot between the various MPO concentrations and the areas under the corresponding luminescence curves of FIG. 9A. FIGS. 9A and 9B suggest that the sensor interface module is capable of detecting even trace amounts of MPO.

Subsequently, the analytical performance of the overall experimental capsule device was also investigated. FIG. 9C shows a sensitivity plot relating voltage to MPO concentration, as recorded through a portable device. As discussed above, the photodetector mounted on the top of the electronic unit detected the gradually increasing luminescence intensity upon increasing concentration (0-9 U/mL) of MPO and converted this photon energy to an electrical signal which was then transmitted to the receiving device through a wireless system embedded inside the electronic unit as described above. As well be appreciated from FIG. 9C, the voltage generated by the photodetector gradually increased with an increase in MPO concentration (0-9 U/mL). Similar to FIG. 9B, FIG. 9D shows a plot between the various MPO concentrations and the areas under the corresponding voltage curves of FIG. 9C. It will be appreciated from the similarity between FIGS. 9B and 9D that the experimental capsule device was capable of detecting even trace amounts of MPO.

### Selectivity Assay

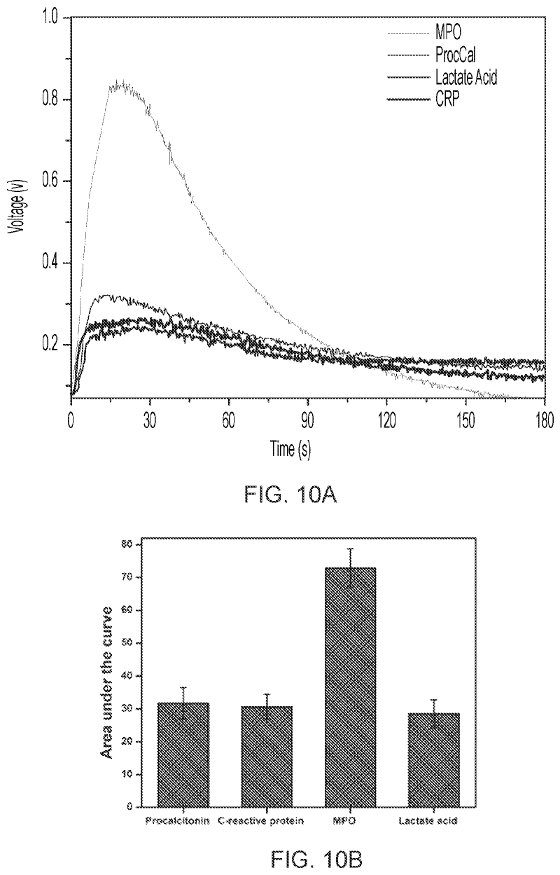

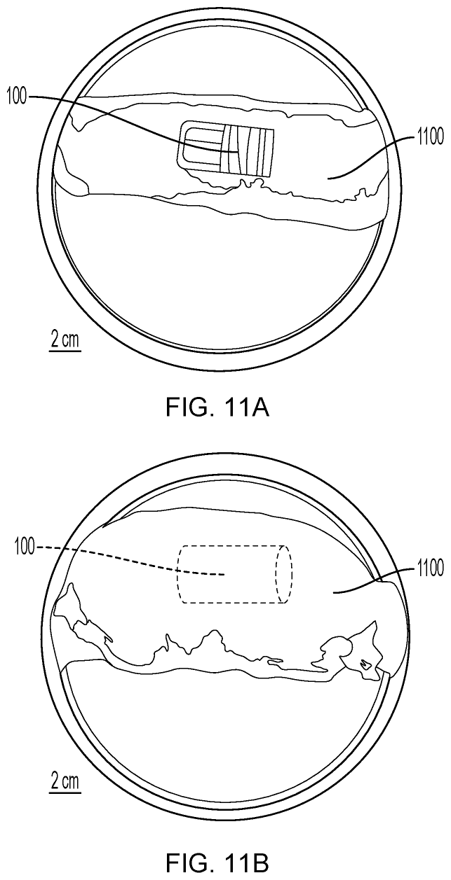

The above results indicate that the experimental embodiment of the disclosed capsule device is capable of detecting MPO. However, the presence of other interfering biomarkers such as procalcitonin, c-reactive protein, and lactic acid may also affect the performance of the device. Therefore, further studies were conducted to evaluate the efficacy of the experimental capsule device in the presence of these biomolecules. FIGS. 10A and 10B illustrate the response of the experimental capsule device in the presence of various interfering biomarkers (each in concentrations of 10 mM). FIG. 10A indicates that the voltage generated by the photodetector in the presence of interfering biomarkers was not significant, while the same concentration of MPO produced a comparatively high output voltage signal. This may be due to the formation of more oxidant (HOCl) by the reaction of UHP and MPO than by the reaction of UHP and any interfering biomarker. The production of less oxidant by these potentially interfering biomolecules resulted in low luminescence intensities and output voltages. FIG. 10B shows a plot between the various interfering biomarkers and the areas under the corresponding voltage curves of FIG. 10A. FIG. 10B indicates that the experimental capsule device was selective and specific to MPO.

### Ex Vivo Detection of MPO

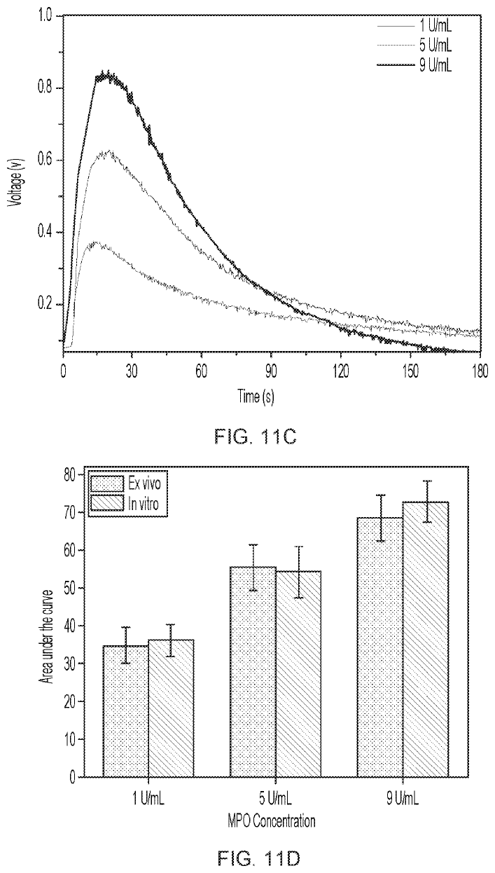

In order to demonstrate the practical application of the experimental capsule device, the ex vivo detection of MPO was also evaluated in the physiological environment as shown in FIGS. 11A and 11B. Here, a porcine small intestine was dissected into three sections 1100 of ˜7 cm in length. Each intestinal section 1100 separately received a solution having a different concentration of MPO: 1, 5, and 9 U/mL. Notably, the three testing concentrations were chosen to compare the analytical performance of the experimental device in both the GI fluid and buffer environments. A fully assembled device 100 was then inserted into each of the intestinal sections 1100 and used to detect the MPO level by recording the voltage signals generated by the photodetector. The resultant voltage signals for each concentration and the area under the curve of each are shown in FIGS. 11C and 11D, respectively. Also shown in FIG. 11D are results of corresponding in vitro buffer experiments. The ex vivo results of FIGS. 11C and 11D, as well as their similarity to the in vitro results, indicate that the experimental embodiment of the capsule device described herein was capable of determining a level of inflammation in the small intestine region by monitoring the level of MPO.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device including one or more processors may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone, tablet, or any other suitable portable or fixed electronic device.

Also, a computing device may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A device for passive sampling of a gastrointestinal tract of a patient, comprising:
a capsule housing bounding a cavity;
a sampling aperture formed in the capsule housing and providing fluid communication between the cavity and an exterior of the capsule housing;
a luminescent substrate layer positioned within the cavity, the luminescent substrate being configured to emit a luminescent light upon exposure to a sample fluid containing a luminescing trigger;
at least one additional substrate layer positioned within the cavity between the sampling aperture and the luminescent substrate, each of the at least one additional substrate layers being configured to chemically interact with the sample fluid;
a photodetector positioned within the cavity, the photodetector configured to detect the luminescent light; and
a biodegradable coating closing the sampling aperture such that degradation of the biodegradable coating exposes the sampling aperture to permit fluid flow into the cavity.

2. The device of claim 1, wherein the luminescing trigger indicates a presence of a biomarker in the sample fluid.

3. The device of claim 2, wherein the biomarker comprises myeloperoxidase (MPO).

4. The device of claim 2, wherein the luminescing trigger comprises an oxidant formed from a chemical interaction with the biomarker.

5. The device of claim 1, wherein the luminescent substrate contains a luminescing agent, and wherein the luminescing agent interacts with the luminescing trigger to emit the luminescent light.

6. The device of claim 5, wherein the luminescing agent comprises luminol.

7. The device of claim 1, wherein one or more of the at least one additional substrate layers is configured to alter a pH of the sample fluid.

8. The device of claim 7, wherein the one or more additional substrate layer configured to alter the pH of the sample fluid contains 4-(Cyclohexylamino)-1-butanesulfonic acid (CABS).

9. The device of claim 1, wherein one or more of the at least one additional substrate layers is configured to interact with the sample fluid to form the luminescing trigger.

10. The device of claim 9, wherein the one or more additional substrate layer configured to interact with the sample fluid to form the luminescing trigger is configured to interact with a biomarker of the sample fluid to form the luminescing trigger.

11. The device of claim 10, wherein the biomarker comprises MPO.

12. The device of claim 10, wherein the luminescing trigger comprises an oxidant.

13. The device of claim 10, wherein the one or more additional substrate layers configured to interact with the sample fluid to form the luminescing trigger contains urea hydrogen peroxide (UHP).

14. The device of claim 13, wherein the one more additional substrate layers containing UHP is infused with a UHP solution containing between 1% and 3% UHP by volume, and wherein the luminescing substrate is infused with a luminol solution containing luminol in a concentration between 20 mM and 30 mM.

15. The device of claim 1, wherein the at least one additional substrate layer comprises a first additional substrate layer configured to alter a pH of the sample fluid and a second additional substrate layer configured to interact with the sample fluid to form the luminescing trigger.

16. The device of claim 1, further comprising a translucent partition positioned between the luminescent substrate and the photodetector, the translucent partition configured to form a fluid-tight seal between a first portion of the cavity and a second portion of the cavity while permitting the luminescent light to pass from the first portion of the cavity to the second portion of the cavity.

17. The device of claim 1, wherein the photodetector comprises an avalanche photodiode.

18. The device of claim 1, further comprising:
a signal processor in communication with the photodetector to receive a detection signal from the photodetector;
a wireless transmitter in communication with the signal processor to transmit a wireless signal, information in the wireless signal being based at least in part on information from the detection signal; and
a battery configured to provide electrical power to at least the signal processor and the wireless transmitter.

19. The device of claim 1, wherein the biodegradable coating comprises a first coating layer and a second coating layer, the first coating layer being configured to degrade within a first pH range and the second coating layer being configured to degrade within a second pH range, the first pH range being different from the second pH range.

20. A method for detecting a biomarker in a gastrointestinal tract of a patient, the method comprising:
administering to a patient an ingestible device comprising:
a capsule housing bounding a cavity,
a sampling aperture formed in the capsule housing and providing fluid communication between the cavity and an exterior of the capsule housing,
a luminescent substrate positioned within the cavity, the luminescent substrate being configured to emit a luminescent light upon exposure to a sample fluid containing a luminescing trigger, the luminescing trigger being indicative of a presence of the biomarker,
a photodetector positioned within the cavity, the photodetector configured to detect the luminescent light and generate a detection signal, and
a wireless transmitter configured to transmit a wireless signal based on the detection signal;
exposing the luminescent substrate to the sample fluid;
receiving the wireless signal at a user device; and
determining, based on the wireless signal, whether the biomarker is present in the sample fluid.

21. The method of claim 20, wherein the biomarker comprises myeloperoxidase (MPO).

22. The method of claim 20, wherein the luminescent substrate contains a luminescing agent, and wherein the luminescing agent interacts with the luminescing trigger to emit the luminescent light.

23. The method of claim 22, wherein the luminescing agent comprises luminol.

24. The method of claim 20, further comprising passing the sample fluid through at least one additional substrate layer of the ingestible device, each additional substrate layer being configured to chemically interact with the sample fluid.

25. The method of claim 24, wherein passing the sample fluid through the at least one additional substrate layer of the ingestible device comprises passing the sample fluid through a first additional substrate layer to alter a pH of the sample fluid.

26. The method of claim 25, wherein the first additional substrate layer contains 4-(Cyclohexylamino)-1-butanesulfonic acid (CABS).

27. The method of claim 24, wherein passing the sample fluid through the at least one additional substrate layer of the ingestible device comprises passing the sample fluid through a second additional substrate layer to form the luminescing trigger.

28. The method of claim 27, wherein the luminescing trigger is an oxidant.

29. The method of claim 27, wherein the second additional substrate layer contains urea hydrogen peroxide (UHP).

30. The method of claim 20, wherein the ingestible device further comprises a biodegradable coating closing the sampling aperture, and wherein exposing the luminescent substrate to the sample fluid comprises degrading the biodegradable coating to permit fluid flow into the cavity.
